(19) United States
(12) Patent Application Publication
(10) Pub. No.: US 2020/0131476 A1
(43) Pub. Date: Apr. 30, 2020

(54) Differentiation Of Immunocytes Using Pluripotent Stem Cells

(71) Applicant: Korea Center For Disease Control And Prevention, Chungcheongbuk-do (KR)

(72) Inventors: Jung Hyun Kim, Seoul (KR); Hyo Won Han, Sejong (KR); Hyang Hee Seo, Chungcheongnam-do (KR); Hyeong Jun Han, Chungcheongbuk-do (KR)

(21) Appl. No.: 16/624,129

(22) PCT Filed: Oct. 12, 2018
(86) PCT No.: PCT/KR2018/012065

§ 371 Date: Dec. 18, 2019

(30) Foreign Application Priority Data
Dec. 11, 2017 (KR) 10-2017-0169750

(51) Int. Cl. C12N 5/0786

(52) U.S. Cl. CPC C12N 5/0645; C12N 2506/03; C12N 2501/155; C12N 2501/2303; C12N 2501/2306; C12N 2501/999; C12N 2501/115; C12N 2501/15; C12N 2500/36; C12N 2501/22; C12N 2501/165; C12N 2501/125; C12N 2501/26; C12N 2506/02; C12N 2501/145

(57) ABSTRACT

The present invention relates to a method for inducing the differentiation of hematopoietic stem cells or macrophages by using pluripotent stem cells, and a composition for inducing the differentiation. Specifically, the present invention relates to a composition for inducing the differentiation of hematopoietic stem cells comprising bone morphogenetic protein 4 (BMP4) in pluripotent stem cells, or a composition for inducing the differentiation of macrophages comprising BMP4 and a macrophage-colony stimulating factor (M-CSF), and a method for inducing the differentiation by using the compositions. The method for inducing the differentiation of the present invention is advantageous in that a differentiation method thereof is simple since only a few types of cytokine are used, and compared to conventional methods for inducing the differentiation, the efficiency of differentiation is improved tens of times or more and the yield of differentiated cells is high.

### TECHNICAL FIELD

The present invention relates to a method for inducing the differentiation of hematopoietic stem cells or macrophages from pluripotent stem cells, and a composition for inducing the differentiation thereof. Differentiated hematopoietic stem cells may be further differentiated into myeloid cells, such as macrophages, neutrophils, monocytes and the like.

The method for inducing the differentiation of the present invention is characterized in that a direct differentiation is carried out without forming an embryonic body (cell aggregate) in a step of differentiating macrophages from pluripotent stem cells. In addition, the method of the present invention uses stepwise treatments of the additives for inducing the differentiation, excludes a composition for inhibiting the differentiation, uses APEL as a basal medium for inducing the differentiation and the like, and thus, has an excellent yield compared to conventional known methods.

### BACKGROUND ART

Pluripotent stem cells are cells at an undifferentiated stage and means stem cells that can differentiate into all of the cell types that make up the human body. The research of the field of stem cells has been launched in earnest since the establishment of human embryonic stem cells (hESCs) in the late 1990s, and has given a quantum leap since the breakthrough of the production of induced pluripotent stem cells (iPSCs) in the mid-2000s. Recently, human somatic-cell nuclear transfer embryonic stem cells (human somatic-cell nuclear transfer ESCs, hSCNT-ESCs) have been also successfully established, and thus, the research of the field of stem cells has become more active.

Among the research fields using such stem cells, various studies are being conducted on the method for differentiating into a desired cell type in cell culture, and in particular, the development of a protocol capable of differentiating it more efficiently are being conducted as a major task. The more the differentiation of stem cells at an undifferentiated stage proceeds, the more the stem cells lose their properties and gradually have intrinsic properties of differentiated cells. In this process, various signal substances, for example, morphogens and growth factors, act sequentially and complexly according to the developmental stage. The differentiation of stem cells is induced by adding these factors to the culture solution of stem cells to be cultured.

Innate immune response is the first line of defense to first protect the body from pathogens invading from the outside. It is also referred to as nonspecific immunity. Neutrophils, monocytes, macrophages and the like are involved in innate immune response. Innate immune response is initiated regardless of the type of pathogens or the presence or absence of infection.

Macrophages are cells responsible for innate immune response and are present in the whole body. Most macrophages are adherent, and include dust cells, microglial cells, Kupffer cells, Langerhans cells and the like. When macrophages recognize an antigen, macrophages ingest the antigen, or secrete toxins to destroy the antigen, or deliver the antigen to lymphocytes to result in an immune response. The macrophages may be present as monocytes that are not differentiated in some blood. Monocytes may be differentiated into dendritic cells or macrophages, if necessary. Various signal substances as described above are involved in the differentiation of macrophages.

As such, the desired cells can be obtained through the induction of the differentiation of pluripotent stem cells. In particular, as in the present invention, the differentiation into immunocytes can be also utilized in the diagnosis of various diseases or in methods for screening drugs. Therefore, there is a need for the development of a novel method for inducing the differentiation of stem cells and a composition for inducing the differentiation of stem cells that can enhance the efficiency of differentiation and the yield of the final cells.

In order to develop a method for inducing the differentiation of hematopoietic stem cells or macrophages with high efficiency by improving the previously developed protocol, the present inventors tried the test of various basal media, the change of the composition of cytokines, sequential regulation of signaling substances and the like. Thus, the present inventors completed the present invention by confirming a method for inducing the successful differentiation from pluripotent stem cells into hematopoietic stem cells followed by into macrophages.

It was confirmed that the macrophages produced by the novel method for inducing the differentiation of the present invention have a very high similarity to macrophages that are present in human, and thus, can be utilized in various ways.

### PRIOR ART DOCUMENT

### Non-Patent Document

Meguma K. Saito et al., Plos One, Published: Apr. 3, 2013 (https://doi.org/10.1371/journal.pone.0059243)

### Patent Document

Korean Patent Publication No. 10-2011-0020468 International Patent Publication No. 2016114723 U.S. Pat. No. 8,372,642

### DETAILED DESCRIPTION OF THE INVENTION

### Technical Problem

It is an object of the present invention to provide a method for inducing effectively the differentiation of myeloid hematopoietic stem cells and macrophages from pluripotent stem cells by regulating various signal substances including bone morphogenetic protein 4 (BMP4) and a macrophage-colony stimulating factor (M-CSF) and the like.

It is another object of the present invention to provide a composition for inducing the differentiation of myeloid hematopoietic stem cells and macrophages derived from pluripotent stem cells that comprises bone morphogenetic protein 4 (BMP4) and a macrophage-colony stimulating factor (M-CSF) for performing the method of the present invention.

### Solution to Problem

In order to solve the above problems, the present invention provides a method for inducing the differentiation of macrophages, comprising a step of maintaining pluripotent stem cells cultured in a mTeSR1 or mTeSR8 basal medium in a dish coated with vitronectin or matrigel; a step of transferring the pluripotent stem cells into a medium for inducing the differentiation of hematopoietic stem cells that comprises BMP4 and culturing the same; a step of treating the pluripotent stem cells with VEGF and SCF and culturing the same, and then, treating the pluripotent stem cells with the signal substances to induce the differentiation of myeloid hematopoietic stem cells; and transferring the differentiated hematopoietic stem cells into a medium for inducing the differentiation of macrophages that comprises M-CSF and culturing the same at a density of 10⁵ cells/cm² or more.

In another embodiment, the present invention provides a composition for inducing the differentiation of macrophages derived from pluripotent stem cells that comprises bone morphogenetic protein 4 (BMP4) and macrophage-colony stimulating factor (M-CSF) on the basis of APEL medium.

Specifically, the present invention provides a method for differentiating into hematopoietic stem cells by treating pluripotent stem cells only with bone morphogenetic protein 4 (BMP4) at a high concentration for 2 days and at a low concentration for 2 days to result in mesoderm induction, and then, treating only with cytokines of VEGF and SCF. The method is characterized in that bFGF and the like that interfere with differentiation are excluded. In addition, the method is characterized in that CDDO methyl ester is further added to further improve the yield of myeloid hematopoietic precursor cells.

The present invention relates to a method for obtaining only pure myeloid hematopoietic precursor cells by treating with cytokines of IL3, IL-6, FLT3, and TPO in order to mature differentiated hematopoietic stem cells into myeloid hematopoietic precursor cells. The method is characterized in that the cells are not treated with M-CSF in the step, and may be an efficient differentiation method in which the yield of pure myeloid hematopoietic precursor cells can be improved in comparison with conventional methods.

The present invention relates to a method for inducing the differentiation comprising a step of inducing the differentiation of macrophages by treating myeloid hematopoietic precursor cells only with a macrophage-colony stimulating factor (M-CSF).

The method for inducing the differentiation of the present invention is characterized in that the efficiency of differentiation is high, and the differentiation method is simple because of a direct differentiation without a step of forming an embryonic body, and the yield of differentiated cells is highest by tens of times or more in comparison with conventional methods.

The macrophages differentiated according to the method for inducing the differentiation of the present invention have a wide range of applications in various fields where a large number of cells are consumed.

### Effect of the Invention

The method for inducing the differentiation of macrophages of the present invention comprises a step of inducing the differentiation from pluripotent stem cells into myeloid hematopoietic stem cells and then inducing the differentiation into macrophages, and uses a method for inducing the differentiation by using various signal substances including bone morphogenetic protein 4 (BMP4) and a macrophage-colony stimulating factor (M-CSF) and the like, and a composition for inducing the differentiation comprising the signal substances. Thus, the method for inducing the differentiation of the present invention is characterized in that a differentiation step is simple, and only a few types of cytokine are used, and the efficiency of differentiation is improved, and the yield of differentiated cells is high.

### BRIEF DESCRIPTION OF DRAWINGS

FIG. 1 is a schematic diagram of the method for inducing the differentiation of myeloid hematopoietic stem cells of the present invention, and is a protocol for the differentiation of hematopoietic stem cells that shows four types of basal media for differentiation of hematopoietic stem cells.

FIG. 2 shows the results of the differentiation of hematopoietic stem cells according to each basal medium by using fluorescence-labeled CD34+ and CD45+, which are markers specifically expressed only in hematopoietic stem cells.

FIG. 3 shows the yield of CD34+CD45+ hematopoietic stem cells (HSPCs) as a quantitative number of cells, which are distinguished by the expression of markers, respectively. This means the number of CD34+CD45+ hematopoietic stem cells produced from 5 colonies.

FIG. 4 shows the yield of CD34+CD45+ hematopoietic stem cells (HSPCs) when treating with a compound CDDO methyl ester in comparison with when not treating with a compound CDDO methyl ester during inducing the differentiation of hematopoietic stem cells.

FIG. 5 shows the ability to differentiate into blood cells of hematopoietic stem cells produced according to the basal medium of the present invention as the ability to form a colony. GM (Granulocyte Macrophage-colony forming unit) shows the ability to differentiate into myeloid blood cells including macrophages. The method for differentiating hematopoietic stem cells of the present invention on the basis of the APEL basal medium mostly has the ability to differentiate into myeloid cells.

FIG. 6 is a schematic diagram of the method for inducing the differentiation of macrophages of the present invention, and is about the whole protocol for inducing the differentiation.

FIG. 7 is a schematic diagram in comparison with the previously known protocol. In comparison with the previously known protocol, bFGF was excluded in step 2, and SCF and M-CSF were excluded and IL6 was added in step 3. In step 4, FL3 and GM-CSF were excluded. The yield of macrophages increased by about 3.8 times on Day 28 in comparison with the previously known protocol, and increased by 100 times when comparing the amounts produced until the end. This indicates that the method of the present invention is a method of increasing the yield while reducing the number of cytokines.

FIG. 8 shows the efficiency of production of macrophages according to the present invention, and shows the expression of macrophage specific markers by labeling with fluorescence when the floating hematopoietic stem cells differentiate into macrophages.

FIG. 9 quantitatively shows the percentage (%), i.e., purity of cells that express macrophage specific markers.

FIG. 10 quantitatively shows the number of macrophages produced from 20 colonies of pluripotent stem cells.

FIG. 11 quantitatively shows the percentage of the cells with fluorescence by analyzing with flow cytometry, as a result of confirming the phagocytosis of the cells through opsonized beads in order to confirm macrophages that completed the differentiation.

FIG. 12 shows the results of analysis of the similarity between differentiated macrophages and human-derived macrophages through a gene expression pattern, wherein the similarity of the gene expression between the differentiated macrophages (iMAC) of the present invention, human monocyte-derived macrophages (hMDM) isolated from human blood, and human macrophage cell lines (Thp-1) is statistically analyzed through principal component analysis (PCA) to confirm the possibility of linkage.

FIG. 13 shows the results of verification of the possibility of infection of differentiated macrophages with virus and bacteria. The differentiated macrophages were infected with H3N2 flu virus or A. Phagocytophilum bacteria, and then, the macrophages were stained with Cytospin method and observed with a microscope.

FIG. 14 shows the results of the observation of the infected macrophages through a TEM (transmission electron microscope) microscope, which shows virus and bacteria in the infected cells.

FIG. 15 shows that ROS increases after the infection of the differentiated macrophages with virus or bacteria.

FIG. 16 shows that the secretion of inflammatory cytokines increases after the infection of the differentiated macrophages with virus or bacteria.

FIG. 17 shows the results of verification of the possibility of infection of the differentiated macrophages with Mycoplasma tuberculosis. After infection, the infection rate was confirmed when treating with Mycoplasma tuberculosis at a MOI concentration of 0 to 20. The infection rate is indicated as an amount of Mycoplasma tuberculosis versus macrophages.

### BEST EMBODIMENT FOR WORKING THE INVENTION

The present inventors studied a method for differentiating pluripotent stem cells into hematopoietic stem cells or macrophages, and completed the method of the present invention, which is characterized in that only a few types of cytokine are used, and the efficiency of differentiation and the yield of macrophages are high.

Specifically, the method for inducing the differentiation of macrophages of the present invention comprises a step of culturing pluripotent stem cells in a mTeSR1 or mTeSR8 basal medium and maintaining the pluripotent stem cells at an undifferentiated stage in a dish coated with matrigel or vitronectin; a step of maintaining the pluripotent stem cells cultured in the basal medium at 5 colonies or less per 35 pi dish; a step of transferring the pluripotent stem cells into a medium for inducing the differentiation of hematopoietic stem cells and culturing the same by treating the pluripotent stem cells only with BMP4 at a high concentration for 2 days and at a low concentration for 2 days on the basis of an APEL culture medium; a step of treating the pluripotent stem cells with VEGF and SCF on the basis of an APEL culture medium and culturing the same, and then, treating with additional signal substances to induce the differentiation into hematopoietic stem cells; and a step of transferring the differentiated hematopoietic stem cells into a medium for inducing the differentiation of macrophages that comprises M-CSF on the basis of a RPMI culture medium and culturing the same at a density of 10⁵ cells/cm² or more.

“bone morphogenetic protein (BMP)” is a peptide growth factor belonging to the transforming growth factor β (TGF-β) superfamily. It is known that BMP plays a role in facilitating the differentiation of stem cells into bone cells or chondrocytes in mammals (see Jiwang Zhang, Linheng Li. BMP signaling and stem cell regulation (2005) Developmental Biology 284 1-11). It was reported that bone inducible BMPs act as the first signal molecules when stem cells differentiate into osteoblasts during a bone morphogenetic process, and in particular BMP2, 4, and 7 are mainly involved in bone formation during a fracture healing process (see M. Egermann, C. A. Lill, and K. Criesbeck, Effects of BMP-2 genetransfer on bone healing in sheep (2006) Gene Therapy, Vol. 13, No. 17, 1290-1299). In addition, it was reported that BMP4 and 6 have a function in inducing bone and cartilage (see Morone Mass., Boden S. Dak., Hair G et al. Gene expression during autograft lumbar spine fusion and the effect of bone morphogenetic protein 2 (1998) Clin Orthop (351) 252; Gruber R, kandler B. Fuerst G et al, Porcine sinus mucosa holds cells that respond to bone morphogenic protein BMP-6 and BMP-7 with increased osteogenic differentiation in vitro (2004) Clin Oral Implants Res 15 (5) 575-580).

The method of the present invention comprise a step of culturing in a medium comprising bone morphogenetic protein 4 (BMP4), which is one of the bone morphogenetic proteins, in order to rapidly differentiate pluripotent stem cells cultured at an undifferentiated stage or dedifferentiated stem cells into mesodermal cells.

BMP4 is a protein involved in a signal transduction pathway inducing the differentiation of pluripotent stem cells into mesodermal cells. The method of the present invention comprise a step of transferring pluripotent stem cells into a medium for inducing the differentiation that comprises BMP4 and culturing the same in order to induce the differentiation of pluripotent stem cells cultured in a basal medium into hematopoietic stem cells, which are mesodermal cells.

A method for differentiating in an induction medium comprising only BMP4 is more efficient than a method for differentiating in a medium comprising Activin A, bFGF, or TGFb, which is the known method. The reason thereof is presumed because cytokines, such as bFGF also act as undifferentiation maintenance factors.

BMP4 may be used at a concentration of 20 to 100 ng/ml, but is not limited thereto. When treating with BMP4 at a concentration of 20 ng/ml or less, inducing the differentiation into mesodermal cells is not performed well, and when treating with BMP4 at a concentration of 100 ng/ml or more for a long period of time, the efficiency is decreased in terms of economy.

In addition, the present invention may include a method for treating with BMP4 at a concentration of 100 ng/ml initially and culturing for 2 days, and then treating at a concentration of 20 ng/ml once more and culturing for 2 days. In the present invention, it is preferable to treat the cells with BMP4 for 4 days and culture the same.

The present invention may include a method of further treating with the CDDO methyl ester compound and culturing in the BMP4 treatment step. When further treating with the CDDO methyl ester compound, the yield of hematopoietic stem cells can be improved by three times or more.

The present invention includes a step of treating with various additional signal substances in order to differentiate pluripotent stem cells, which are induced into mesodermal cells, into myeloid hematopoietic stem cells on a medium for inducing the differentiation.

The signal substances that may be used in the present invention may include vascular endothelial growth factor (VEGF), stem cell factor (SCF), platelet growth factor (thrombopoietin, TPO), interleukin-6 (IL-6), interleukin-3 (IL-3), and FMS-like tyrosine kinase (FMS-like tyrosine kinase 3, Flt3).

In the present invention, the signal substances may be sequentially included in the induction medium.

When treating with the signal substances for the induction of the differentiation simultaneously, the sufficient differentiation is not achieved, and when treating with BMP4 and then with other signal substances sequentially, the differentiation is induced more effectively. This is because the process of inducing the differentiation of pluripotent stem cells into mesodermal cells by the treatment with BMP4 has an important effect on the whole process for inducing the differentiation.

In one embodiment of the present invention, the method comprises a step of culturing for 2 days by treating with VEGF and SCF, and then, culturing for 10 days or more by further treating with TPO, IL6, IL3 and Flt3 to differentiate into myeloid hematopoietic stem cells. The above step comprises further treating with the signal substances necessary for the differentiation of hematopoietic stem cells. VEGF and SCF play a role in facilitating the initial differentiation (hemangioblast). TPO, IL6, IL3 and Flt3, which are added sequentially in the treatment step, help to play a necessary role not only in the differentiation, but also in the self-proliferation of hematopoietic stem cells.

When comparing the method of treating with VEGF, SCF, TPO, IL3, IL6, and Flt3 simultaneously with the method of treating with VEGF and SCF for 2 days and then treating with TPO, IL6, IL3, and Flt3, the latter method is more effective for the differentiation of hematopoietic stem cells.

The above signal substances are necessary substances for the differentiation of hematopoietic stem cells. It is preferable that the signal substances are sequentially added in the treatment step in order to differentiate into mesodermal cells and then differentiate into hematopoietic stem cells.

The hematopoietic stem cells of the present invention have properties as GMP (granulocyte-macrophages progenitor), which is an intermediate stage in the differentiation into macrophages, megakaryocytes, or neutrophils. The induction of this differentiation is closely influenced by the kind of the above signal substances and the order of treatment. The production of GMP occurs most largely when the differentiation is induced under the above conditions.

The basal medium used in the present invention may be APEL (albumin polyvinylalcohol essential lipids), which is an animal serum-free and animal-derived component-free medium. APEL is a medium that Andrew G. Elefanty first developed for using in the production of embryonic bodies from embryonic stem cells (Nature protocol 3, 768-776, 2018). APEL comprises the composition of Table 1 below.

TABLE 1
Composition of APEL medium

1 x Iscove's modified Dulbecco's medium (IMDM)
1 x Ham's F-12 nutrient mixture
Albucult (rh Albumin) (5 mg/ml)
Deionized BSA (2.5 mg/ml)
Polyvinylalcohol (PVA)
Linoleic acid (100 ng/ml)
Linolenic acid (100 ng/ml)
SyntheChol (synthetic cholesterol) (2.2 μg/ml)
α-Monothioglycerol (α-MTG) (3.9 μl per 100 ml; ~350-450 μM)
rh Insulin-transferrin-selenium-ethanolamine solution (rhITS-Eth)
Ascorbic acid 2 phosphate (50 μg/ml)
Glutamaxl (L-alanyl-L-glutamine) (2 mM)
Penicillin/streptomycin (50 U Pen G/50 μg streptomycin sulfate)

In addition, the method of the present invention comprises a step of differentiating macrophages from the differentiated hematopoietic stem cells. In the step of the present invention, hematopoietic stem cells may be treated with macrophage-colony stimulating factor (M-CSF) to differentiate into macrophages. In the above step, the cells differentiated into hematopoietic stem cells are maintained and cultured as much as possible, and then treated with M-CSF to differentiate into macrophages. In the above step, the cells differentiated into GMP among hematopoietic stem cells are treated with M-CSF.

One embodiment of the present invention comprises a step of treating with M-CSF and IL-3 simultaneously or sequentially. When treating with IL-3 first, the differentiation rate of myeloid hematopoietic stem cells may be increased. After that, treating with M-CSF results in the generation of macrophages with higher purity since myeloid hematopoietic stem cells mostly differentiate into macrophages, and has a much higher yield compared to the previously known differentiation methods.

In addition, the method of treating with M-CSF and IL-3 sequentially has a higher yield in comparison with the method of treating with M-CSF and IL-3 simultaneously.

In the above step, the concentration of M-CSF may be initially 100 ng/ml. After a certain period of time, the concentration of M-CSF may be reduced to 20 ng/ml.

When macrophages are differentiated from pluripotent stem cells according to the method for inducing the differentiation of the present invention, hematopoietic stem cells are induced to have the properties as GMP, and GMPs are obtained as much as possible before treating with M-CSF, and then, GMPs may be differentiated into macrophage at once. Thus, the yield of macrophages may be increased.

The present invention relates to a method for inducing the differentiation of hematopoietic stem cells, comprising 1) a step of culturing pluripotent stem cells in a medium for inducing the differentiation of hematopoietic stem cells that comprises BMP4; 2) a step of culturing the pluripotent stem cells by treating the pluripotent stem cells with VEGF and SCF; and 3) a step of treating the pluripotent stem cells with TPO, IL-6, IL-3, and Flt3.

In step 1) of the method for inducing the differentiation of the present invention, any one or more of Activin A, bFGF, and TGFb is not further comprised, so that the number of cytokines to be used can be reduced.

In addition, in step 1), it is preferable to comprise BMP4 at a concentration of 20 to 100 ng/ml, and it may be preferable to treat with BMP4 at a low concentration initially and then treat with BMP4 at a high concentration once again.

In addition, the medium for inducing the differentiation of hematopoietic stem cells is preferably APEL (albumin polyvinylalcohol essential lipids).

The present invention relates to a method for inducing the differentiation of macrophages, comprising 1) a step of culturing pluripotent stem cells in a medium for inducing the differentiation of hematopoietic stem cells that comprises BMP4; 2) a step of culturing the pluripotent stem cells by treating the pluripotent stem cells with VEGF and SCF; 3) a step of inducing the differentiation of hematopoietic stem cells by treating the pluripotent stem cells with TPO, IL-6, IL-3, and Flt3; and 4) a step of culturing the differentiated hematopoietic stem cells by adding the differentiated hematopoietic stem cells to a medium for inducing the differentiation of macrophages that comprises M-CSF.

It is preferable that the cell density is maintained at 1×10⁵ cells/cm² or more when culturing macrophages in step 4).

The present invention relates to hematopoietic stem cells differentiated from pluripotent stem cells, wherein the differentiation is induced by the method for inducing the differentiation.

The present invention relates to macrophages differentiated from pluripotent stem cells, wherein the differentiation is induced by the method for inducing the differentiation.

The present invention relates to a composition for inducing the differentiation of hematopoietic stem cells derived from pluripotent stem cells, comprising one or more selected from the group consisting of bone morphogenetic protein 4 (BMP4), vascular endothelial growth factor (VEGF), stem cell factor (SCF), platelet growth factor (thrombopoietin, TPO), interleukin-6 (IL-6), interleukin-3 (IL-3), CDDO methyl ester, and FMS-like tyrosine kinase (FMS-like tyrosine kinase 3, Flt3).

In addition, the present invention relates to a composition for inducing the differentiation of macrophages derived from pluripotent stem cells, comprising one or more selected from the group consisting of bone morphogenetic protein 4 (BMP4), vascular endothelial growth factor (VEGF), stem cell factor (SCF), platelet growth factor (thrombopoietin, TPO), interleukin-6 (IL-6), interleukin-3 (IL-3), FMS-like tyrosine kinase (FMS-like tyrosine kinase 3, Flt3), and macrophage-colony stimulating factor (M-CSF).

In the present invention, the term “pluripotent stem cell” is a stem cell having a differentiation totipotency, and a stem cell capable of differentiating into an endodermal, mesodermal, or ectodermal cell or a tissue. The origin of a cell includes, but is not limited to, a human embryonic stem cell and a dedifferentiated stem cell.

In the present invention, the term “mesodermal cell” refers to a cell whose differentiation is induced by signal molecule components that regulate the differentiation of stem cells, in particular by the BMP4 signal transduction pathway. The mesodermal cells of the present invention may include mesodermal cells whose differentiation is regulated by BMP4, and may include all the cells that are differentiated into hematopoietic mesodermal cells through mesodermal cells.

In the present invention, the term “hematopoietic stem cell” is a hematopoietic cell that can be potentially differentiated into a major component of blood, and is also referred to as “hemangioblast.” Hematopoietic cells can be divided into the cells generated in the fetal liver of the developmental stage, the cells generated in the yolk sac, and the cells produced in the postnatal bone marrow, and are differentiated into lymphoid cells and myeloid cells. The hematopoietic stem cells of the present invention can be differentiated into macrophages.

In the present invention, the term “macrophage” is responsible for innate immunity, and it exists in the form of monocytes in the blood, and can be differentiated into dendritic cells or macrophages. Macrophages play a role in removing bacteria or virus, which is carried out by phagocytosis. The macrophages differentiated through the process of inducing the differentiation of the present invention can be utilized for a study model of a viral or bacterial infection.

In the present invention, the term “differentiation” means a process in which an unspecialized cell develops into a specific cell, and in particular, includes a process of developing from a stem cell into a specific cell. In the present invention, embryonic stem cells and dedifferentiated stem cells are used as cells having the ability to differentiate, and the embryonic stem cells and dedifferentiated stem cells can be finally differentiated into macrophages through mesodermal cells and hematopoietic stem cells.

In the present invention, the term “signal substance” is a concept including all of signal proteins, cytokines, catalysts and the like involved in the differentiation of pluripotent stem cells. Particularly, in the present invention, a signal substance includes bone morphogenetic protein (BMP), macrophage-colony stimulating factor (M-CSF), vascular endothelial growth factor (VEGF), stem cell factor (SCF), platelet growth factor (TPO), IL-6, IL-3, and Flt3.

In the present invention, CDDO methyl ester is an Nrf2 activation substance, and means “2-cyano-3,12-dioxooleana-1,9(11)-dien-28-oic acid methyl ester” compound.

Hereinafter, the present invention will be described in more detail with reference to examples. These examples are only for describing the present invention more specifically, and the scope of the present invention is not limited by these examples.

### WORKING EXAMPLE

### Example 1

Protocol for Differentiation of Hematopoietic Stem Cells

1) Pluripotent stem cells were adapted to a matrigel-coated dish and a mTeSR1 medium for 3 weeks, and then other colonies except for 5 colonies in 35 pi dish were removed by pipetting. This is a process for providing space for the proliferation of differentiated cells in the future. At this time, when vitronectin is used as a coating material or mTeSR8 is used as a medium, the differentiation of hematopoietic stem cells or macrophages can be induced.

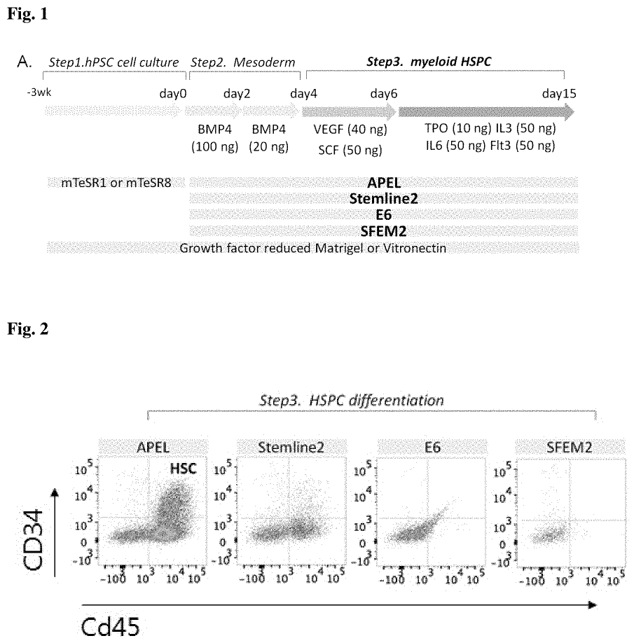

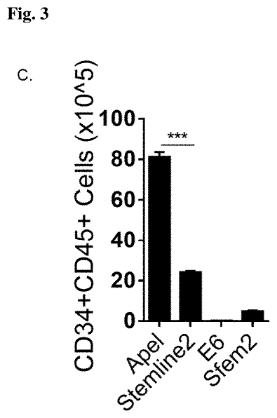

2) The medium for culturing the stem cells used in 1) above was replaced with APEL medium. As shown in FIGS. 2 and 3, this is because the efficiency of differentiation of the hematopoietic stem cells of the APEL medium is much higher than that of other basal media. The differentiation into mesodermal cells was induced rapidly by treating with BMP4 at a high concentration (100 ng/ml) for 2 days and then sequentially at a low concentration (20 ng/ml) for 2 days.

In addition, the efficiency of differentiation of hematopoietic stem cells can be increased by treating with the CDDO methyl ester compound together with BMP4 for 4 days.

In order to differentiate the mesodermal cells into initial hematopoietic stem cells (hemangioblasts), the mesodermal cells were then treated with VEGF and SCF, followed by with IL-3, IL-6, TPO, and Flt3 to induce the differentiation into the hematopoietic stem cells. The reason why the signal substances were not simultaneously used in the treatment is to increase the efficiency of differentiation and purity.

The protocol for the differentiation is illustrated in detail in FIG. 1.

### Example 2

Confirmation of Expression of Marker for Hematopoietic Stem Cells

CD34+ and CD45+ are markers specifically expressed in hematopoietic stem cells. The mesodermal cells differentiated from pluripotent stem cells were induced to differentiate into hematopoietic stem cells again. In order to confirm this, the amount of expression of the markers specifically expressed in hematopoietic stem cells was measured.

The differentiated cells were monolayered and reacted with the antibodies CD34 and CD45 labeled with fluorescence. Then, FACS (flow cytometry) was used to confirm the cells that present antigens on each of their surfaces. The results are shown in FIGS. 2 and 3.

As shown in FIGS. 2 and 3, CD34-positive and CD45-positive cells were confirmed, respectively. The results show that the differentiation into hematopoietic stem cells progressed.

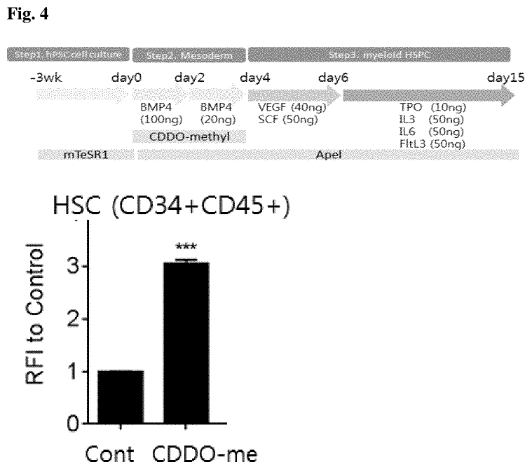

In addition, as shown in FIG. 4, it was found that when 50 nM of the CDDO methyl ester compound was added together with BMP4 for 4 days to induce the differentiation, the efficiency of differentiation of hematopoietic stem cells was increased by about 3 times.

### Example 3

Confirmation of Ability of Myeloid Hematopoietic Stem Cells

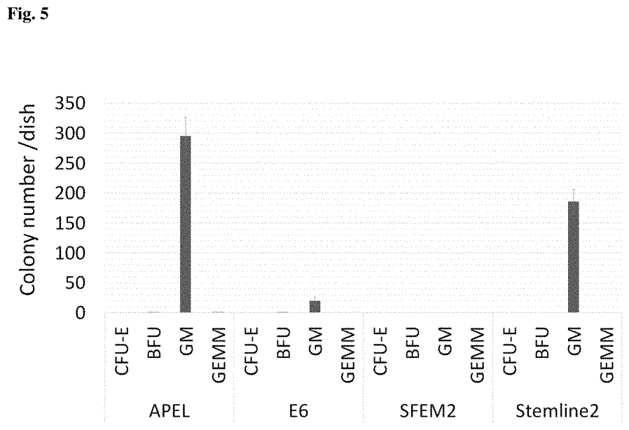

The colony forming assay was carried out to confirm the ability to form blood cells of the differentiated hematopoietic stem cells. The results are shown in FIG. 5, and it was confirmed that most of GM (granulocyte-macrophage) colonies were formed. As shown in FIG. 5, it was found that the hematopoietic stem cells produced in Example 1 were myeloid hematopoietic stem cells.

### Example 4

Protocol for Differentiation of Macrophages

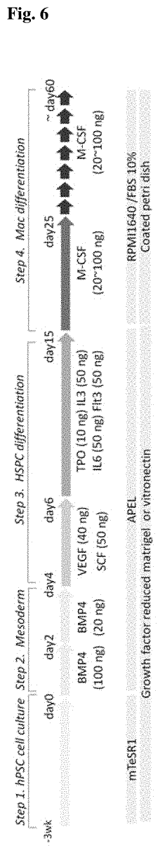

1) The hematopoietic stem cells (floating cells) were collected, transferred to a fresh 60 pi dish (a coated general cell culture dish), and treated with 100 ng/ml of M-CSF alone to induce the differentiation into macrophages. RPMI1640 was used as a basal medium, and 10% FBS was added. After culturing for 10 days, the cells were sub-cultured at a ratio of 1:2. At this time, the density of the cells is very important. When the cells are maintained at a density of 10⁵ cells/cm² or more on the plate, the yield of macrophages can be increased. At this time, the concentration of M-CSF in the treatment can be 20 to 100 ng/ml. The protocol for the differentiation is shown in detail in FIG. 6.

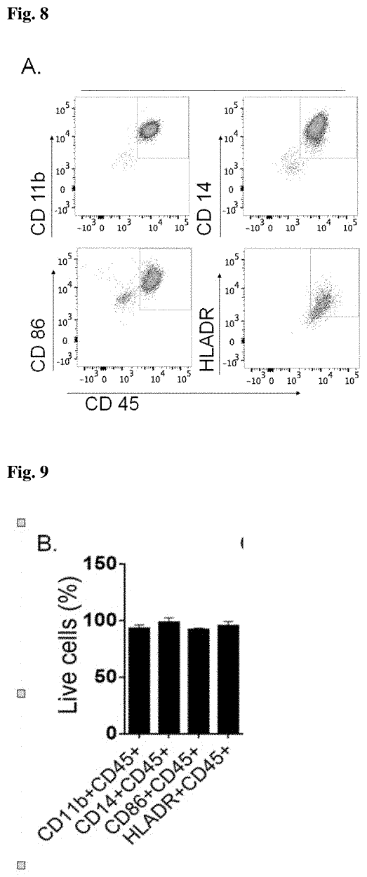

As shown in FIGS. 8 and 9, it was confirmed that when sub-culturing, macrophages were continuously produced for 60 days or more. In addition, it was confirmed that 98% or more of the produced macrophages were CD14+, CD11b+, CD45+, CD86+ cells and had the properties of macrophages.

The differentiated macrophages were cultured in a RPMI1640 medium and a medium containing 10% FBS. It was observed that the amount of production was increased by orbital shaking during culturing.

### Example 5

Confirmation of Expression of Marker for Macrophages

CD14, CD11b, and CD86 are markers specifically expressed in macrophages. The differentiation of the differentiated hematopoietic stem cells into macrophages was induced. In order to confirm this, the amount of expression of the markers was measured. The results are shown in FIG. 8. As shown in FIG. 8, it was confirmed that 98% or more of the macrophages expressed CD14, CD11b, and CD86 and CD45, which is a marker in blood.

It was found that the purity of the macrophages differentiated from the pluripotent stem cells produced in Example 4 above was 98% or more.

The total number of macrophages produced by the differentiation method and the composition for inducing the differentiation of the present invention was 5×10⁸ or more, which was produced from 20 colonies of pluripotent stem cells.

### Example 6

Comparison of Yield of Differentiation of Macrophages

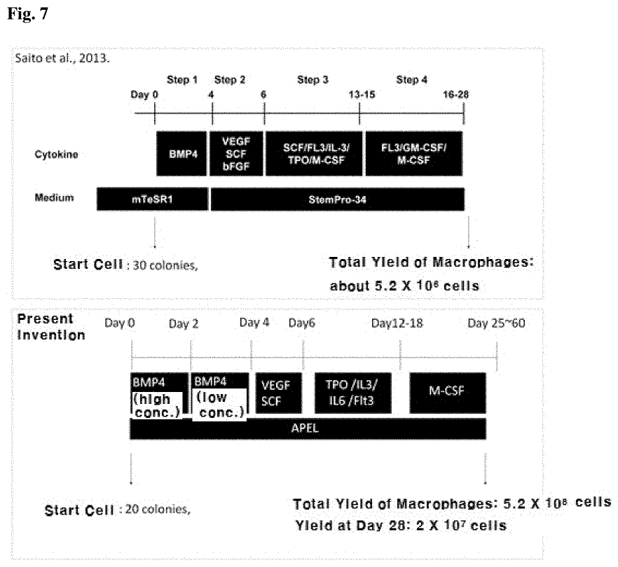

The above differentiation protocol was compared with a previously known protocol for the differentiation of macrophages derived from pluripotent stem cells (eguma K. Saito et al., Plos One, Published: Apr. 3, 2013), and the results are shown in FIG. 7. That is, in comparison with the above conventional differentiation protocol, the differentiation protocol of the present invention excluded bFGF in step 2, excluded SCF and M-CSF and added IL-6 in step 3, and excluded FL3 and GM-CSF in step 4. It was shown that the total yield of macrophages obtained by the differentiation protocol of the present invention was higher by 100 times than that of the conventional differentiation protocol.

### Example 7

Phagocytosis of Differentiated Macrophages

In order to confirm the phagocytosis of the differentiated macrophages by the protocol of the present invention, the experiment was carried out by the following method.

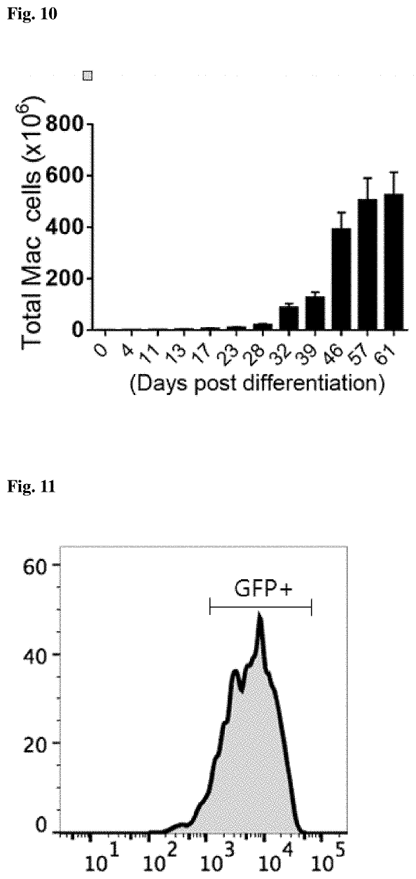

Human serum was used as a sample, and the differentiated macrophages were added into opsonized beads, and the phagocytosis was confirmed. As a result, it was confirmed that the phagocytosis occurred actively. As shown in FIG. 11, it was confirmed that 95% or more of the CD11b-positive and CD14-positive macrophages were involved in the phagocytosis. It was found that the differentiated macrophages performed their normal functions.

### Example 8

Confirmation of Similarity Between Macrophages of the Present Invention and Human-Derived Macrophages

In order to confirm and compare the similarity between the macrophages produced in Example 4 and human-derived macrophages, the experiment was carried out by the following method.

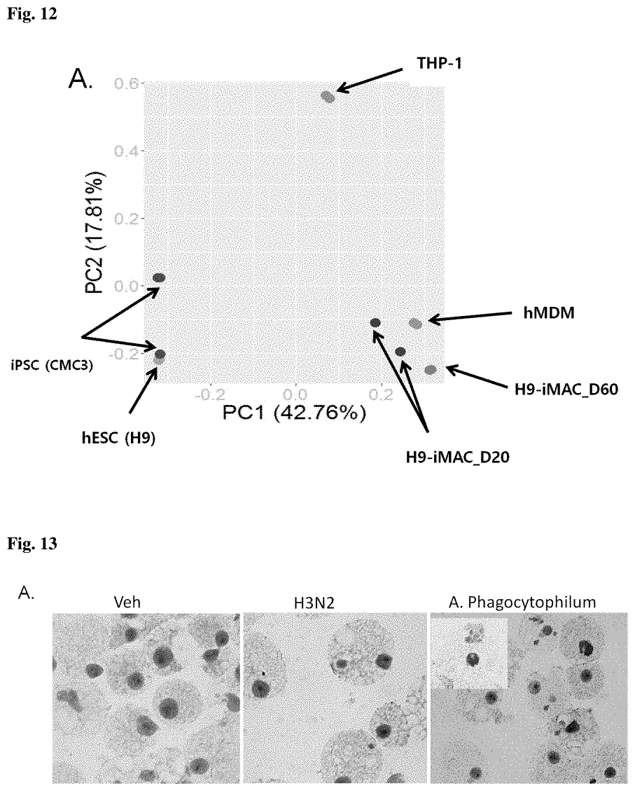

The genomic expression similarity between the differentiated macrophages (20 days after differentiation, 40 days after differentiation), human macrophages Thp 1 cell line, human blood cell-derived macrophages, which were obtained by drawing the human blood, and purifying PBMCs (periperal blood monocyte cells), and then, isolating only monocytes from CD14+ macrophages by using a CD14+ isolation kit, and pluripotent stem cell lines was confirmed by a RNAseq analysis, and the results are shown in FIG. 12. As shown in FIG. 12, it was found that the genomic expression level of the differentiated macrophages are highly similar to that of the human blood cell-derived macrophages.

### Example 9

Confirmation of Infection Function of Macrophages and Utilization of Research of Infectious Diseases

In order to confirm the possibility of infection of the macrophages produced in Example 4 with virus and bacteria, the confirmation test was carried out by the following method using a real influenza virus or Anaplasma bacteria infection model. That is, 10⁶ cells of the differentiated macrophages were infected with influenza virus H3N2 (1MOU), and then, cultured at 37° C. for 1 to 7 days. The results are shown in FIG. 13.

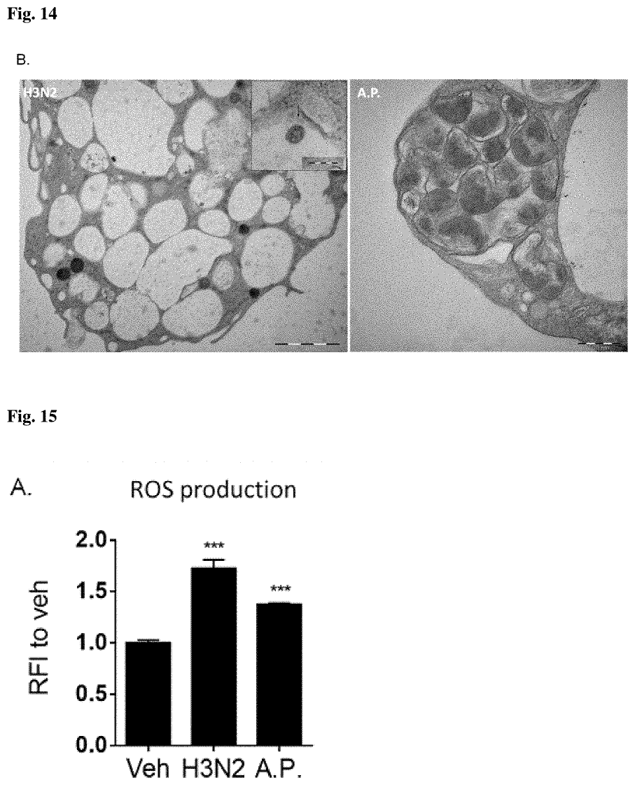

H3N2 virus infection in cells stained with Cytospin was confirmed at the macroscopic level. In addition, Morula of Anaplasma was also confirmed in the differentiated macrophages. This result is more clearly confirmed by the transmission electron microscopy, and is shown in FIG. 14.

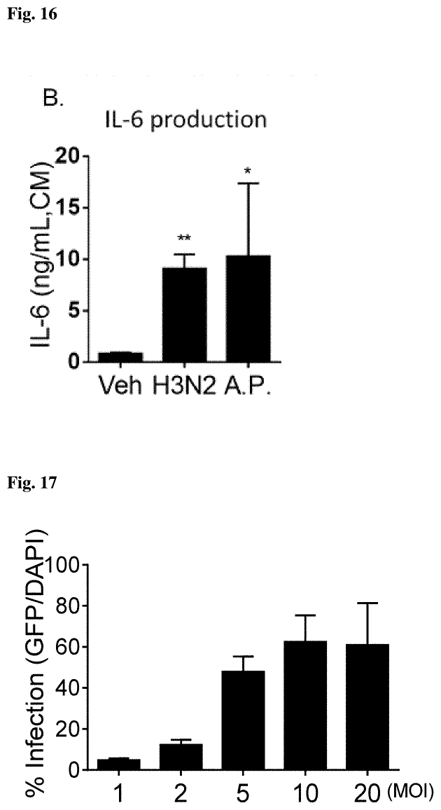

In addition, as shown in FIGS. 15 and 16, it was confirmed that ROS increased from macrophages after a viral or bacterial infection, and the amount of secretion of inflammatory cytokine IL-6 increased.

### Example 10

Research of Mycoplasma tuberculosis Infection Using Macrophages

In order to confirm whether the macrophages produced in Example 4 above can be used as a study model of a bacterial infection, the following experiment was carried out. Specifically, the macrophages were infected with Mycoplasma tuberculosis, and then, cultured in a 37° C. 5% CO₂ incubator.

20,000 macrophages were infected with Mycoplasma tuberculosis at a MOI concentration of 1 to 20, and the results are shown in FIG. 17. As shown in FIG. 17, it was confirmed that the macrophages infected with Mycoplasma tuberculosis were increased.

Mycoplasma tuberculosis used herein was Mycoplasma tuberculosis into which the GFP vector was inserted, and thus, Mycoplasma tuberculosis was labeled with fluorescence. In order to confirm the infection rate of macrophages, the macrophages were stained with DAPI. Mycoplasma tuberculosis indicated by GFP and macrophages indicated by DAPI were observed by confocal microscopy, and the infection rate of Mycoplasma tuberculosis within macrophages was quantitated.

### Example 11

Confirmation of Similarity Between Differentiated Macrophages During Infection and Human Macrophages

In order to confirm whether the differentiated macrophages similarly response to human macrophages during infection, the differentiated macrophages (iMAC) and human blood-derived macrophages (hMDM) were infected with Mycoplasma tuberculosis MO15. After 5 days, the pattern of change of gene expression was confirmed by RNAseq. As a result, it was confirmed that the change of gene expression of the differentiated macrophages (iMAC) for Mycoplasma tuberculosis was similar to human blood-derived macrophages (hMDM).

1. A method for inducing the differentiation of hematopoietic stem cells, characterized in that the method comprises
1) a step of culturing pluripotent stem cells in a medium for inducing the differentiation of hematopoietic stem cells that comprises BMP4;
2) a step of culturing the pluripotent stem cells by treating the pluripotent stem cells with VEGF and SCF; and
3) a step of treating the pluripotent stem cells with TPO, IL-6, IL-3, and Flt3.

2. The method for inducing the differentiation of hematopoietic stem cells according to claim 1, characterized in that step 1) comprises further treating with CDDO methyl ester.

3. The method for inducing the differentiation of hematopoietic stem cells according to claim 1, characterized in that the medium in step 1) does not further comprise any one or more of Activin A, bFGF, and TGFb.

4. The method for inducing the differentiation of hematopoietic stem cells according to claim 1, characterized in that BMP4 in step 1) is comprised at a concentration of 20 to 100 ng/ml.

5. The method for inducing the differentiation of hematopoietic stem cells according to claim 1, characterized in that step 1) comprises treating with BMP4 at a low concentration followed by treating with BMP4 at a high concentration once again.

6. The method for inducing the differentiation of hematopoietic stem cells according to claim 1, characterized in that the medium for inducing the differentiation of hematopoietic stem cells is APEL (albumin polyvinylalcohol essential lipids).

7. A method for inducing the differentiation of macrophages, comprising
1) a step of culturing pluripotent stem cells in a medium for inducing the differentiation of hematopoietic stem cells that comprises BMP4;
2) a step of culturing the pluripotent stem cells by treating the pluripotent stem cells with VEGF and SCF;
3) a step of inducing the differentiation of hematopoietic stem cells by treating the pluripotent stem cells with TPO, IL-6, IL-3, and Flt3; and
4) a step of culturing the differentiated hematopoietic stem cells by adding the differentiated hematopoietic stem cells to a medium for inducing the differentiation of macrophages that comprises M-CSF.

8. The method for inducing the differentiation of macrophages according to claim 7, characterized in that the cell density is maintained at 1×10⁵ cells/cm² or more when culturing macrophages in step 4).

9. Hematopoietic stem cells differentiated from the pluripotent stem cells, characterized in that the differentiation is induced by the method for inducing the differentiation according to claim 1.

10. Macrophages differentiated from the pluripotent stem cells, characterized in that the differentiation is induced by the method for inducing the differentiation according to claim 7.

11. A composition for inducing the differentiation of hematopoietic stem cells or macrophages derived from pluripotent stem cells, comprising one or more selected from the group consisting of bone morphogenetic protein 4 (BMP4), vascular endothelial growth factor (VEGF), stem cell factor (SCF), platelet growth factor (thrombopoietin, TPO), interleukin-6 (IL-6), interleukin-3 (IL-3), CDDO methyl ester, FMS-like tyrosine kinase (FMS-like tyrosine kinase 3, Flt3) and macrophage-colony stimulating factor (M-CSF).

12. The composition according to claim 11, for inducing the differentiation of macrophages derived from pluripotent stem cells, comprising one or more selected from the group consisting of bone morphogenetic protein 4 (BMP4), vascular endothelial growth factor (VEGF), stem cell factor (SCF), platelet growth factor (thrombopoietin, TPO), interleukin-6 (IL-6), interleukin-3 (IL-3), FMS-like tyrosine kinase (FMS-like tyrosine kinase 3, Flt3), and macrophage-colony stimulating factor (M-CSF).

13. The composition according to claim 11, for inducing the differentiation of hematopoietic stem cells derived from pluripotent stem cells, comprising one or more selected from the group consisting of bone morphogenetic protein 4 (BMP4), vascular endothelial growth factor (VEGF), stem cell factor (SCF), platelet growth factor (thrombopoietin, TPO), interleukin-6 (IL-6), interleukin-3 (IL-3), CDDO methyl ester, and FMS-like tyrosine kinase (FMS-like tyrosine kinase 3, Flt3).
